(19) United States
(12) Patent Application Publication
(10) Pub. No.: US 2025/0352061 A1
(43) Pub. Date: Nov. 20, 2025

(54) METHODS AND SYSTEMS FOR CONTINUOUS ANALYTE MONITORING

(71) Applicant: Abbott Diabetes Care Inc., Alameda, CA (US)

(72) Inventors: Anup J. SHAH, Portland, OR (US); Paul R. E. JARVIS, Pudsey (GB); Matthew BATES, New Hope, PA (US); Naveen THURAMALLA, Alameda, CA (US)

(21) Appl. No.: 19/209,194

(22) Filed: May 15, 2025

Related U.S. Application Data
(60) Provisional application No. 63/648,104, filed on May 15, 2024.
(60) Provisional application No. 63/648,597, filed on May 16, 2024.

(51) Int. Cl. A61B 5/00; A61B 5/145

(52) U.S. Cl. CPC A61B 5/0004; A61B 5/0022; A61B 5/14546; A61B 5/7405; A61B 5/742; A61B 5/746

(57) ABSTRACT

A continuous analyte monitoring system, includes a continuous analyte monitor, the continuous analyte monitor configured to generate analyte data based on the patient's measured analyte levels, the continuous analyte monitor having a transmitter configured to transmit the analyte data. In some embodiments, the continuous analyte monitoring system is a continuous lactate monitoring system. The system also includes an external transmitter that is configured to receive the lactate data from the transmitter and a computing device connected to the continuous analyte monitor through the external transmitter. The continuous analyte monitoring system is implemented within a distributed diagnostic system that generates customized visual elements for alerts and notifications that are communicated to multiple distributed downstream recipient devices.

### CROSS-REFERENCE TO RELATED APPLICATIONS

This application claims the benefit of U.S. Provisional Application No. 63/648,104, filed May 15, 2024, and U.S. Provisional Application No. 63/648,597, filed May 16, 2024, both of which are incorporated herein by reference in their entirety.

### FIELD

The subject matter of this disclosure generally relates to systems, devices, and methods for improving diagnosis systems using data from continuous analyte monitoring systems and providing for customizable visual elements, alerts, and notifications. More specifically, this disclosure relates to improved diagnosis capability in conjunction with a continuously connected graphical user interface system for dynamically generating alerts and notifications based on continuous analyte data.

### BACKGROUND

This disclosure relates to the field of continuous analyte monitoring in the context of a distributed monitoring and/or diagnosis system that interconnects different diagnosis devices, such as patient devices, health care provider devices, electronic health record (EHR) systems, remote monitoring systems, and caregiver devices. In some embodiments, the distributed diagnosis system provides continuous lactate monitoring and utilizes continuous lactate data within the system. The use of continuous analyte data enables the real-time monitoring and notification that provides improved monitoring, diagnostic and communication capabilities between the interconnected devices of the diagnosis system.

Currently, lactate is measured using serial blood draws, typically in a hospital or other health care setting. These measurements are often reactive in nature because they are usually taken in response to patient issues (e.g., a patient presenting with certain symptoms) or at a given point in time. This results in analysis of lactate levels and any potential corresponding treatment just before or as a patient's medical condition deteriorates. This is because current methodologies do not constantly monitor patient conditions and use the monitored information to predict potential patient outcomes, which could lead to earlier identification of deterioration and earlier administration of preventative and/or curative treatment.

Lactate is an analyte in which in vivo levels may vary in response to numerous environmental or physiological factors including, for example, eating, physiological stress, exercise, sepsis or septic shock, heart failure, hypoxia, and the like. In the case of chronic or ongoing conditions, periodic laboratory measurements of lactate levels may be sufficient to determine whether these conditions are increasing or decreasing in severity, and/or if the patient is responding to treatment. Other lactate-altering conditions may be episodic in nature, in which case lactate levels may fluctuate very rapidly and irregularly. Other use-cases in which continuous analyte information, including lactate, may be used for predicting patient outcomes include patients in a hospital setting, patients in a home setting, disease prediction and detection such as sepsis and heart failure, and patients that have undergone certain kinds of surgical procedures. Conventional laboratory measurements may be ill suited to determine lactate levels in such instances. Namely, lactate levels may have changed several times between successive measurements, and an abnormal lactate level may be completely missed in such instances, thereby leading to potentially incorrect diagnoses. In the case of rapidly fluctuating lactate levels, it can be desirable to measure an individual's lactate levels continuously, such as through using an implanted in vivo lactate sensor. Even if a lactate spike is observed when measuring lactate levels with periodic laboratory measurements, there often is no possibility of taking proactive actions to alleviate or remediate a particular condition leading to the elevated lactate levels. This can have significant consequences for a user's health and well-being in some cases.

### BRIEF SUMMARY OF THE INVENTION

This disclosure describes continuous lactate monitoring as an exemplary implementation for a continuous monitoring system within a distributed diagnostic system. However, the systems and methods of receiving, processing, and displaying the lactate data are generally applicable to other forms of analyte data. Thus, other analytes may be utilized, either in combination with lactate (e.g., a dual sensor continuous monitoring system) or independently on its own. Other examples of analytes that can be utilized within this distributed diagnostic system include, but are not limited to, glucose, alcohol, ketone, potassium, NT-proBNP, sodium, L-DOPA, and creatinine.

A continuous lactate monitoring system includes a continuous lactate monitor connected to the patient, the continuous lactate monitor configured to generate lactate data based on measured lactate levels of the patient lactate, the continuous lactate monitoring having a transmitter configured to transmit the lactate data; an external transmitter that is configured to receive the lactate data from the transmitter; a computing device connected to the continuous lactate monitor through the external transmitter, wherein the computing device includes a processor and memory, the memory storing instructions that when executed by the processor cause the processor to: receive lactate data from the continuous lactate monitor; analyze the lactate data to compare the data to an alarm condition; and transmit a notification and/or an alert if the lactate data triggers the alarm condition.

The memory can include further instructions that cause the processor to transmit a notification and/or an alert to a second computing device when the alarm condition is triggered.

The alert may be an audible, haptic, and/or visual alert. The notification may be transmitted by email, text message, internal message, and/or other data integration modality (e.g., through use of an application programming interface).

The memory can include a user preference associated with a user of the computing device, the user preference related to the alarm condition. The alarm condition can include one or a combination of exceeding a threshold limit and maintaining an analyte level over the threshold limit (i.e., state) for a defined period of time (e.g., an hour).

The user preference may also be stored on a remote server such that the user preference can be retrieved by the computing device using a data network.

The memory may include further instructions that cause the processor to transmit the lactate data to a remote server using a data network.

The system may include a second computing device configured to receive the lactate data from the remote server.

A method of monitoring lactate in a patient includes providing a continuous lactate monitor to be connected to the patient; generating lactate data based on measuring the patient's lactate levels using the continuous lactate monitor; transmitting the lactate data to a computing device using a transmitter connected to the continuous lactate monitor; analyzing the lactate data to compare the data to an alarm condition; and transmitting a notification and/or an alert if the lactate data triggers the alarm condition.

The method may further include transmitting a notification to a second computing device when the alarm condition is triggered.

The method may further include storing a user preference associated with a user of the computing device in a memory of the computing device, the user preference related to the alarm condition.

The method may further include storing the user preference on a remote server such that the user preference can be retrieved by the computing device using a data network.

the method may further include transmitting the lactate data from the computing device to a remote server using a data network.

The method may further include receiving the lactate data from the remote server at a second computing device.

Certain aspects of the disclosure have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS/FIGURES

The accompanying drawings, which are incorporated herein and form a part of the specification, illustrate the present disclosure and, together with the description, further serve to explain the principles thereof and to enable a person skilled in the pertinent art to make and use the same.

FIG. 1 is a system and data flow diagram of a continuous lactate monitor (“CLM”) system.

FIG. 2 is a system data flow diagram of a CLM system.

FIG. 3 is a diagram of activation of a continuous lactate monitor in a healthcare setting.

FIG. 4 is a diagram of activation of a continuous lactate monitor in a home setting.

FIG. 5 is a data flow diagram of a distributed diagnostic system using continuous analyte data.

FIG. 6 is a system diagram of a CLM system used in a home environment.

FIG. 7 is a system diagram of a CLM system used in a healthcare environment.

FIG. 8 is a flow diagram of a CLM system activation process using a device in a healthcare environment.

FIG. 9 is a diagram of data and alert pages available in a CLM system in a healthcare environment according to an embodiment.

FIG. 10 is a flow diagram of a CLM system activation process using an external transmitter device in a healthcare environment.

FIG. 11 is a flow diagram of a CLM system data functionality on a patient bedside monitor in a healthcare environment.

FIG. 12 is a flow diagram of a CLM system activation process in a home environment.

FIG. 13 is a flow diagram of a patient induction process in an emergency department setting including CLM system activation.

FIG. 14 is a flow diagram of a patient induction process in an intensive care unit setting including CLM system activation.

FIG. 15 is a flow diagram of a patient induction process in a general care floor setting including CLM system activation.

FIG. 16 is a data flow diagram of a CLM system.

In the drawings, like reference numbers generally indicate identical or similar elements. Additionally, generally, the left-most digit(s) of a reference number identifies the drawing in which the reference number first appears.

### DETAILED DESCRIPTION

Reference will now be made in detail to systems and methods illustrated in the accompanying drawings. When a particular feature, structure, or characteristic is described in connection with an example, it is submitted that it is within the knowledge of one skilled in the art to affect such a feature, structure, or characteristic in connection with other examples whether or not explicitly described.

Lactate is a bi-product of human metabolism that is present in the human body. Lactate levels that exceed predetermined ranges can indicate a variety of different abnormal conditions in a patient. Current practice is for lactate to be measured using a standard blood draw in a healthcare setting, which provides only a single measurement at one point in time. These measurements are also usually reactive in that the lactate measurement is usually ordered when the patient is already in the healthcare setting with other symptoms. This means that elevated lactate measurements are typically not useful in predicting therapy needs in a patient in advance. Further, the single point measurement cannot capture transient fluctuations in lactate. Finally, the limited lactate data reduces the ability to leverage data analysis tools and alerts that can improve patient outcomes by helping Health Care Professionals (“HCPs”) better understand a patient's metabolic health. Thus, there exists a need for improved lactate monitoring and data analysis.

Continuous lactate monitoring systems employ an insertable or implantable sensor, which detects and monitors blood lactate levels. As such, these systems can be referred to as “in vivo” monitoring systems. The sensor can be part of the continuous lactate monitor that resides on the body of the user. The monitor can contain the electronics and power supply that enable and control the lactate sensing. Typically, an applicator is employed to insert the sensor in the body of the patient.

The invention relates to a system that includes a continuous lactate monitor connected to the patient. The continuous lactate monitor is configured to repeatedly and periodically record the patient's lactate levels. The continuous lactate monitor has an external transmitter that is connected to a computing device. The computing device includes a processor and memory, the memory storing instructions that when executed by the processor cause the processor to receive lactate data from the continuous lactate monitor. Further instructions cause the processor to analyze the lactate data to compare the data to an alarm condition and cause the processor to transmit a notification and/or an alert if the lactate data meets the alarm condition.

Benefits of this disclosure include the ability to continuously analyze lactate data to identify trends or transient variations in the lactate data. This can result in abnormal conditions being identified earlier, which can improve patient outcomes by enabling earlier treatment initiation. Further, as will be discussed below different alerts and alarms based on data analysis can also identify conditions that would not be apparent with a single reading. Further, systems and methods discussed here are intended for use outside of a healthcare environment, which means these conditions can be identified without requiring the patient to visit a HCP.

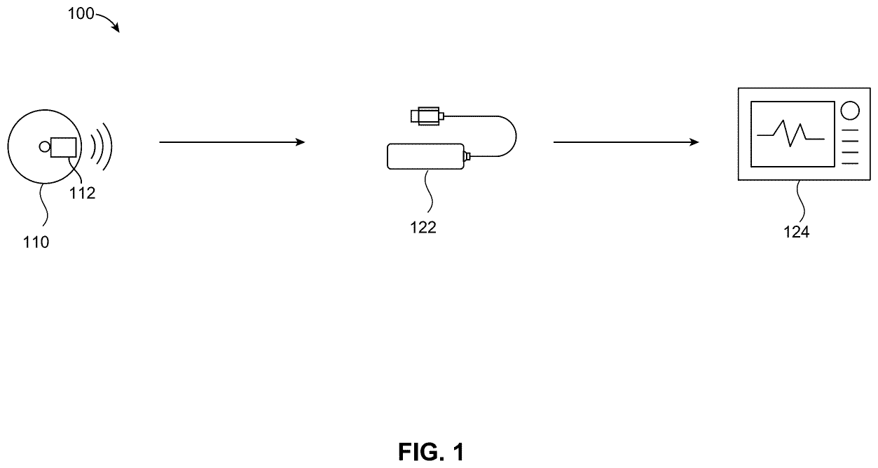

FIG. 1 is a system diagram of a continuous lactate monitoring system 100. In some embodiments, system diagram may include other continuous analyte monitoring systems in addition to or instead of continuous lactate monitoring system 100. For example, other continuous analyte monitoring may include monitoring glucose, alcohol, ketone, potassium, NT-proBNP, sodium, L-DOPA, and creatinine. A continuous lactate monitor 110 is configured to be connected to a patient for measuring the patient's lactate. As discussed below, the continuous lactate monitor 110 may be removably disposed on the patient's skin. Continuous lactate monitor 110 is designed to repeatedly measure the lactate in a patient at either regular or irregular intervals. For example, continuous lactate monitor 110 can repeatedly measure the lactate at a predetermined interval, such as every minute, five minutes, or ten minutes. This frequency can be changed by a suitable command received at continuous lactate monitor 110. Continuous lactate monitor 110 may also measure lactate “on demand.” On demand measurements are based on continuous lactate monitor 110 receiving an instruction to measure lactate, for example from a separate device operated by user as discussed below. Continuous lactate monitor 110 includes suitable processing and memory capability to record and store the lactate data readings as required.

Continuous lactate monitor 110 also includes a transmitter 112 that enables continuous lactate monitor 110 to communicate with a remote computing device 120 using an external transmitter 122. Transmitter 112 and external transmitter 122 may be configured to communicate wirelessly through a suitable wireless communication protocol, such as Bluetooth, Bluetooth Low Energy, Wi-Fi, NFC, for example. In addition or alternatively, transmitter 112 and external transmitter 122 may include a wired connection.

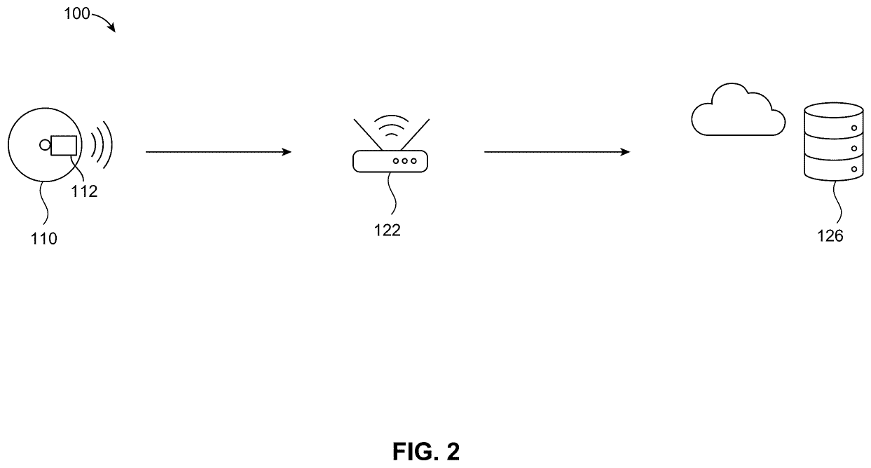

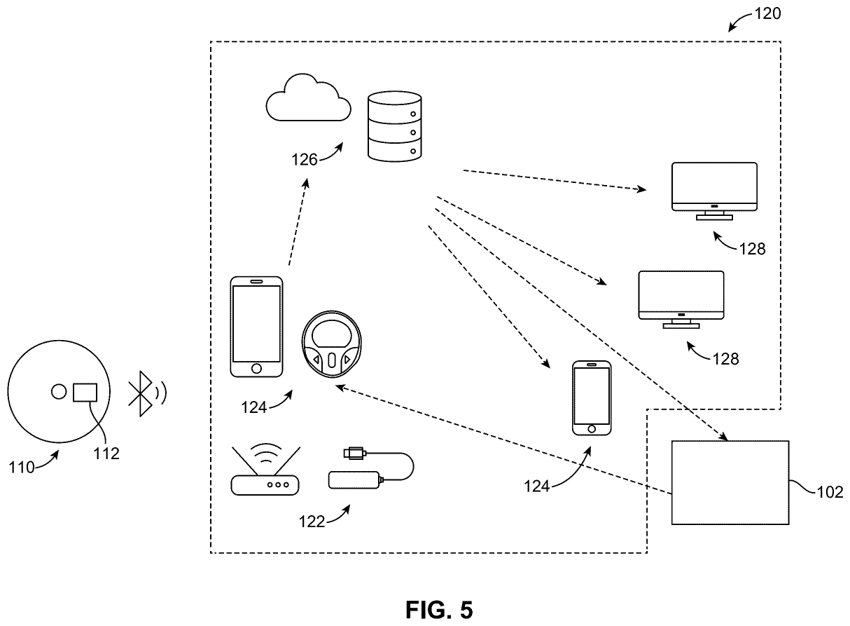

Remote computing device 120 can include any number of computing devices that are spatially separate from continuous lactate monitor 110. For example, remote computing device 120 can include one or more reader devices 124 (see FIG. 2), a gateway (e.g., a Bluetooth gateway), one or more remote servers 126, and/or one or more display devices 128 (see FIG. 5) each having suitable processor and memory capabilities. Reader devices 124, remote servers 126, and display devices 128 may be directly connected to or physically incorporate external transmitter 122, or may be indirectly connected to the external transmitter 122, for example by other suitable data transmitters, computing devices, servers, or other similar structures. Reader devices 124 may be a dedicated device for interfacing with continuous lactate monitor 110, or may be a separate computing device running an application that allows the computing device to interface with continuous lactate monitor. For example, reader device 124 may be a mobile phone or tablet running an application. Remote servers 126 can be any physically separated computing device that is configured to send and receive data using a suitable data network. Display devices 128 may be devices that can receive and display lactate data. It should be noted that reader devices 124 may also be considered display devices if they include display capabilities. Any of these reader devices 124, remote servers 126, and display devices 124 can include a display that can be used to visualize lactate data and alarm notifications. The reader devices 124 and remote servers 126 can include an input mechanism such as a keyboard or touchscreen to input commands.

FIG. 5 depicts a data flow diagram of a distributed diagnostic system using continuous analyte data. The distributed diagnostic system is configured with real-time alarms capability based on its receipt of continuous analyte data from multiple continuous analyte sensors. Because it has access to continuous analyte data from multiple sources (e.g., different patients, different hospitals), the distributed diagnostic system is configured to detect trends and tracks patterns for identifying and predicting disease progression. The distributed diagnostic system is designed to reliably and securely transmit and receive analyte measurement data and generate customized visual elements for configuring alerts and notifications that are transmitted to digitally connected devices. The use of continuous lactate data provides better adherence to disease (e.g., sepsis) protocols. Real-time data eliminates blind spots within distributed diagnostic systems, detects deterioration and accelerates treatment decisions, leading to better patient outcomes. This detection can be supported by third party monitoring services 102, which can receive the lactate data and continuously monitor it for issues. The third party monitoring service 102 can, in turn, contact the patient, caregiver, and/or HCP directly to alert them of the issue, as shown by the arrows in FIG. 5. Note that this communication can be facilitated by the lactate monitoring application running on computing devices 120, which can include messaging services. In addition or alternatively, messaging can be accomplished by other suitable techniques (e.g., phone, email, text messaging).

External transmitter 122 can be configured as an adapter that enables communication between continuous lactate monitor 110 and an existing computing device 120 or multiple computing devices 120. This can be accomplished, for example, by configuring external transmitter 122 with an interface that is compatible with the existing computing device, such as a universal serial bus connection. In this way existing computing devices can be leveraged to become reader devices 124 without requiring replacement of the existing computing device. For example, external transmitter 122 can be plugged into an existing patient bedside monitor or central monitoring station in a hospital, which can then be used to access lactate data from continuous lactate monitor 110 via external transmitter 122. A single external transmitter 122 may be sufficient to pass lactate data in a hospital setting because of the existing data network in place. For example, patient bedside monitors are already configured to share data with a central monitoring station and/or the patient's electronic health record. Thus, external transmitter 122 need only be integrated with the corresponding patient bedside monitor to allow sharing of the lactate data with other hospital computing systems such as the central monitoring station. External transmitter 122 may act only as a pass-through device for any lactate data, and will not retain any of that data in any memory once the data has been forwarded. In other embodiments, external transmitter 122 can have memory to retain lactate data and an integrated display that allows a user to view lactate data retained on and/or received by external transmitter 122. In some of these embodiments, a user can also the display in combination with suitable input elements (e.g., buttons and/or a touchscreen) to control operation of external transmitter 122. Note that external transmitter 122 can simultaneously interface with multiple computing devices 120, including through the use of different communication protocols (e.g., a wired connection and a wireless connection).

After the continuous lactate monitor 110 has warmed up post-activation and is ready to transmit lactate data, individual data packets may be transmitted to external transmitter 122. External transmitter 122 (e.g., a Bluetooth Gateway) may be located within Bluetooth communication range of the continuous lactate monitor 110. Individual data packets may contain (at a minimum) measurement value, sensor serial number, and time stamp. Individual data packets may be transmitted at a predetermined frequency (e.g., once every one minute). Note that individual computing devices 120 can each have their own customized frequency at which they receive data. This customized frequency may differ from the data reception frequency from continuous lactate monitor 110. This customized frequency may also be modified based on various factors, including patient condition. For example, if a patient's condition is stable, the frequency may be reduced to reduce the data transmission requirements. If a patient condition is deteriorating, the frequency may be increased to ensure adequate coverage of the patient's lactate measurements. This change in frequency can be manually input by an HCP or other user, or may be automatically updated based on lactate data analysis using techniques discussed below.

External transmitter 122 may be configured to approve continuous lactate monitor 110 to be able to receive data packets from continuous lactate monitor 110, for example, over Bluetooth. External transmitter 122 may be configured to communicate with a single continuous lactate monitor 110. In addition or alternatively, external transmitter 122 may be configured to communicate with multiple continuous lactate monitors 110 concurrently. External transmitter 122 may be configured to optimize the number of monitors connected to it based on an optimization process. The optimization process may ensure that data transmitted to external transmitter 122 is accurately received and processed. External transmitter 122 may self-determine a threshold number of monitors based on the optimization process.

Once sensor data (from a single monitor or from multiple monitors simultaneously) is received by remote computing device 120, it can send the data to downstream locations, such as other remote computing devices. The remote computing device 120 can send the sensor data to downstream locations over one or more wired and/or wireless communication methods (e.g., Wi-Fi, cellular, radio frequency). Examples of other remote computing devices include a cloud server (i.e., cloud data management (“DM”) account), an on-premise server (i.e., on-premise DM account), a hospital cloud server, hospital on-premise server, and/or other cloud or on-premise server.

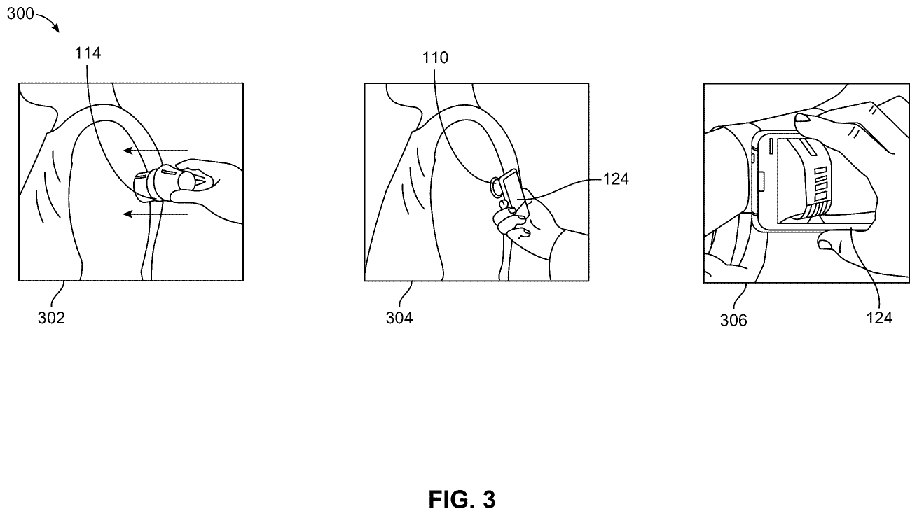

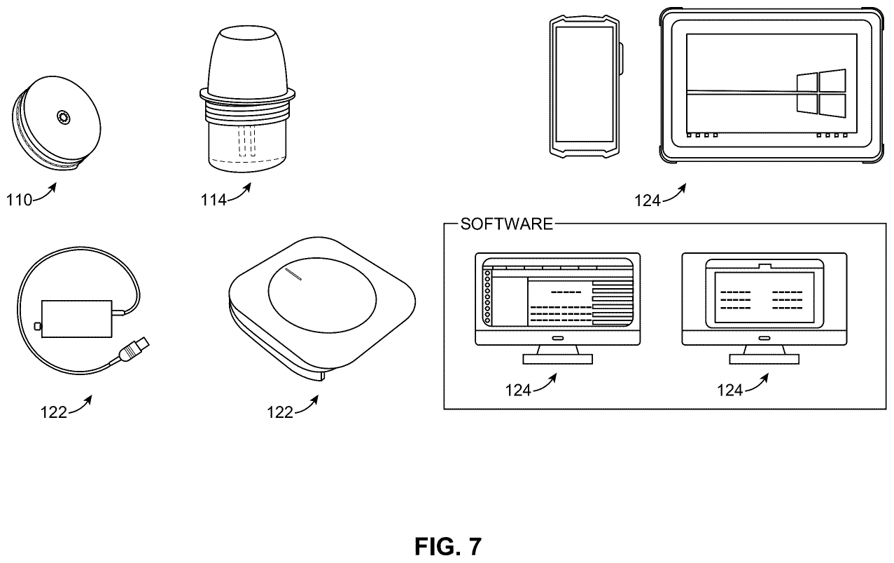

FIG. 3 is a flow diagram of an application process 300 for applying and initializing continuous lactate monitor 110 in a healthcare environment, such as a hospital. FIG. 7 is a system diagram of components of the corresponding system. In a step 302 continuous lactate monitor 110 is fixed to the patient using an applicator 114. This step also includes positioning a sensor beneath the patient's skin to enable sensing of lactate. In a step 304 continuous lactate monitor 110 is activated by using reader device 124, typically by the HCP installing continuous lactate monitor 110. This activation may occur via wireless transmission using transmitter 112 to communicate with reader device 124, and initializes continuous lactate monitor 110 for use. Note that the activation may occur using a different wireless communication protocol than used by transmitter 112 for data transmission. For example, activation may be accomplished using a Near Field Communication protocol while lactate data transmission may occur using Bluetooth. A prerequisite to step 304 may require the HCP to verify their ability to activate continuous lactate monitor 110. This can ensure that the HCP is trained and authorized to activate continuous lactate monitor 110. For example, reader device 124 may require the HCP to login to their user account before allowing activation of continuous lactate monitor 110. Alternatively, the HCP may need to input a code or password into reader device 124 instead of logging into a user account. In a step 306 the activation of continuous lactate monitor 110 is linked to identifying information of the patient. For example, in embodiments like FIG. 3 the HCP may use reader device 124 to input patient identifying information. In FIG. 3 this input process is illustrated as scanning an identification band of the patient using a camera reader device 124. The HCP may input the patient information into reader device 124 manually, for example by using a keyboard or touchscreen. Or reader device 124 may already be linked to the patient information, for example through a database that the HCP has logged into. In this example step 306 can happen automatically after step 304 is performed because the patient information is already present in reader device 124.

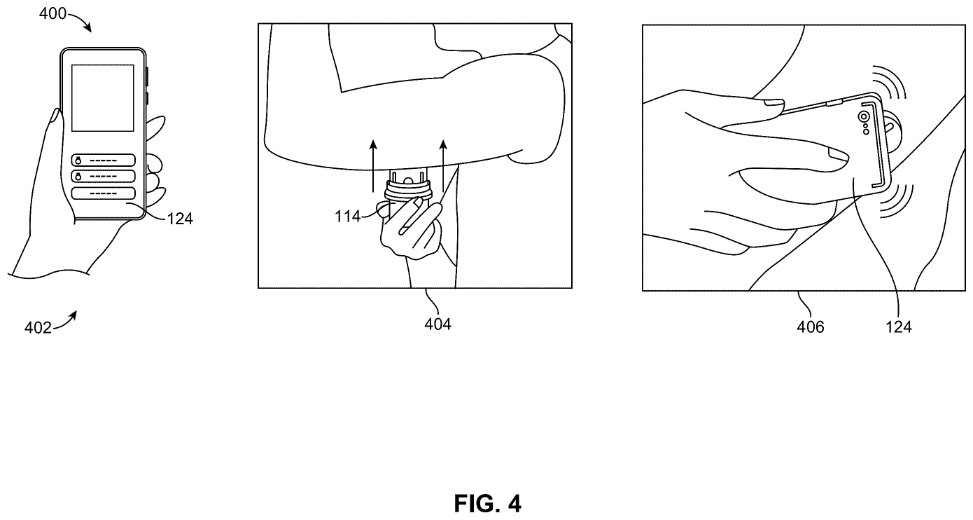

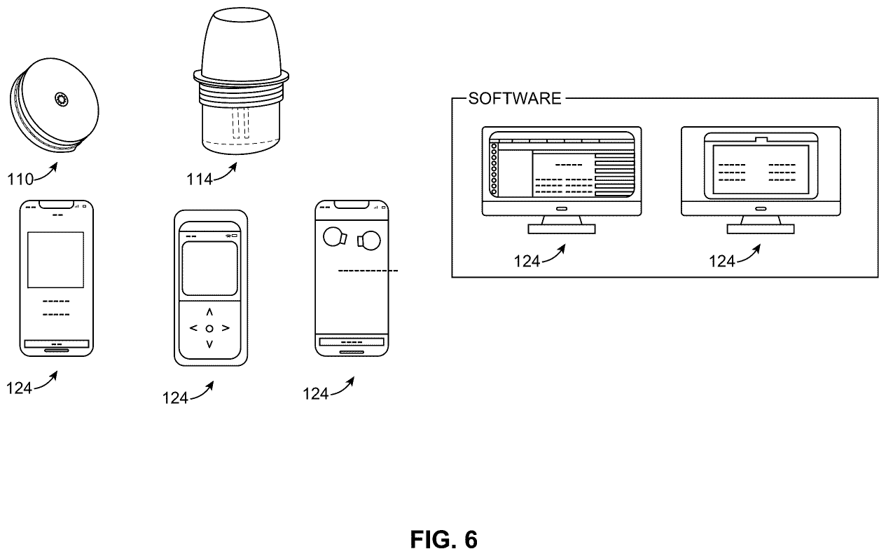

FIG. 4 is a flow diagram of an application process 400 for applying and initializing continuous lactate monitor 110 in a home environment, which can be any non-healthcare environment that a patient may be in, such as a home, workplace, or temporary accommodation. FIG. 6 is a corresponding system diagram of components available at home. This process begins with a verification step 402 where the patient verifies their information on reader device 124. This may occur when the patient logs into a user account on reader device 124. This user account is associated with their information. Step 404 is similar to step 302 and results in continuous lactate monitor 110 being applied to the patient via applicator 114. Step 406 is similar to step 304 above and involves using reader device 124 to activate continuous lactate monitor 110. Because the patient has already logged in to an account in verification step 402 continuous lactate monitor 110 is associated with the patient's information automatically.

In the home setting, continuous lactate monitor 110 may be implemented on the back of the patient's arm (e.g., by the patient or caregiver). In the home setting, the continuous lactate monitor 110 may communicate with reader devices 124 and/or remote servers 126 for identifying conditions associated with readmission optimization and home monitoring. For example, one or more reader devices 124 may be co-located with the continuous lactate monitor 110 and one or more reader devices 124 may be located remote from continuous lactate monitor 110. Data communicated from continuous lactate monitor 110 may be forwarded to each of the one or more reader devices 124 for identifying threshold conditions associated with making post-discharge decisions which include generating alerts and notifications with regard to readmissions and discharge.

Once implemented, the continuous lactate monitor 110 may be activated using a wireless communication protocol (e.g., near field communication (NFC), radiofrequency, Bluetooth). The activation may be performed using an authorized reader device that may be installed with an application (customized for the user, e.g., a patient app) that allows activation of the monitor and receiving data and alerts from the continuous lactate monitor 110. NFC activation may be a two-way communication between the continuous lactate monitor 110 and the application. The application receives continuous lactate data from continuous lactate monitor 110 including the sensor serial number. Once continuous lactate monitor 110 has been applied and activated, it must then be associated to a patient's identity, in order for the lactate data to be tied to a specific patient.

In a home setting, the distributed diagnostic system is configured to provide customized visual elements to authorized devices that are based on the continuous lactate data provided by continuous lactate monitor 110. Other configurable aspects of the system include frequency that data is communicated and recipient devices authorized to monitor and have access to the data.

In order to associate a continuous lactate monitor 110 to a patient's identity in the home setting, the application may be configured to authorize a user into their DM account. The DM account may be authorized by a third party and secured so that the application is only accessible to a particular user with an authorized reader device. Examples of authorization techniques include use of a secure access link, a QR code that provides a secure access link, a pre-registered user email address, or a unique username and password combination. Once the application has been logged into the patient's DM account, any continuous lactate monitors 110 (i.e., may be more than one) that are activated using the application may communicate individual data packets. The individual data packets may contain at least a measurement value, sensor serial number, and time stamp. Additional data packets may contain information related to an alarm condition being met. Individual data packets may be transmitted at a predetermined frequency (e.g., once every one minute). Once lactate data is received by the application, the application can send the data to remote computing device 120 (e.g., a cloud server) and/or reader device 124 (or multiple examples thereof), where it is stored and made available to send to multiple downstream locations.

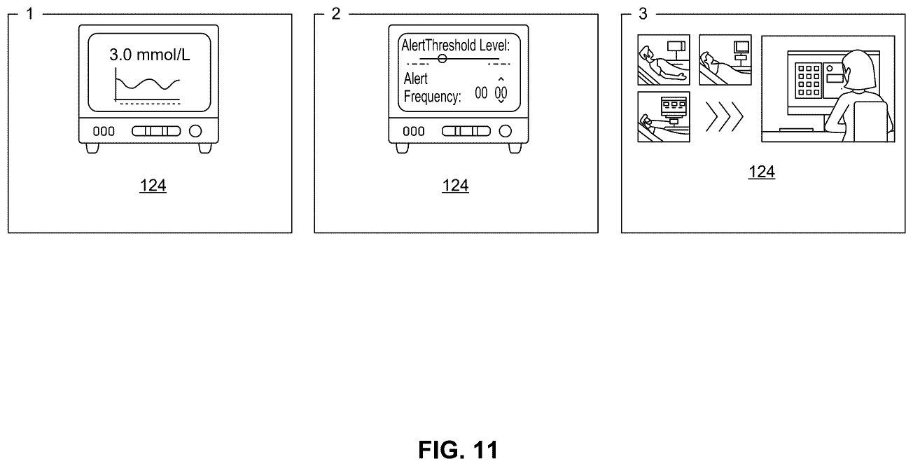

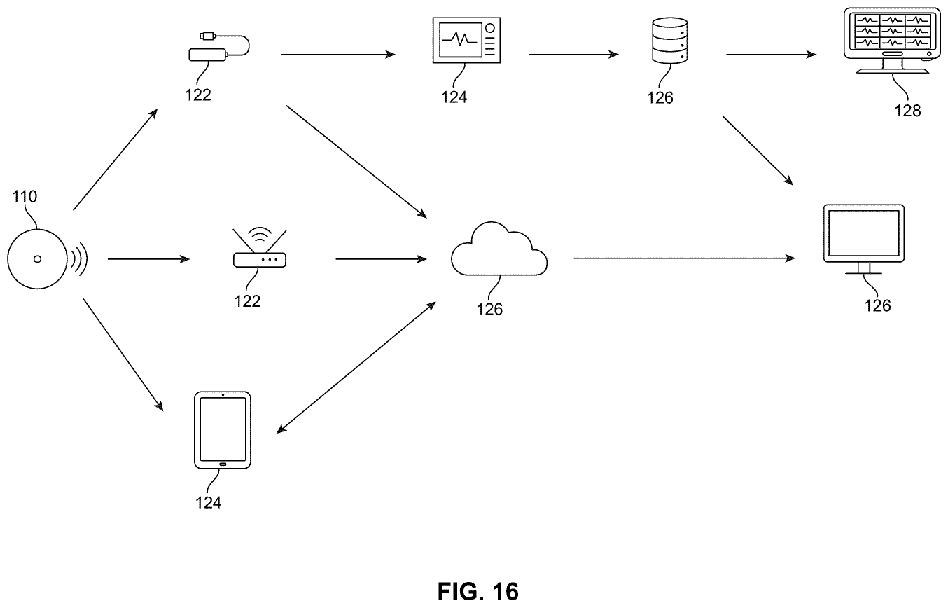

As discussed above, there may be multiple remote computing devices 120 that are able to receive lactate data from continuous lactate monitor 110. FIGS. 5 and 16 show an example of continuous lactate monitor 110 being connected to several reader devices 124, remote servers 126 (FIG. 16), and display devices 128 via suitable data transmission networks. Some examples of reader devices 124 include existing patient monitors, mobile phones, tablets, or centralized monitoring systems. Examples of display devices 128 includes computing devices such as computers, mobile phones, or tablets belonging to an HCP or third party service. The lactate data can also be transmitted directly to the patient's electronic health record for storage and access purposes. This system allows for increased flexibility and continuity in accessing patient data because different HCPs who have appropriate data access can review the patient's lactate data on their own reader device 124 as needed. This flexibility extends to situations where the patient starts lactate monitoring at home and then moves to a healthcare facility because the data from home is maintained and can be accessed at a later time at the healthcare facility. The opposite situation is also a benefit of this system because the HCP can maintain continuity of lactate monitoring of the patient after discharge from a hospital back to a home environment. This can be especially useful in early detection of relapses or unexpected degradation in patient condition that may warrant re-admission to a hospital or healthcare environment. An example of this flexibility is shown in FIG. 11, which has different examples of reader devices 124, including a patient bedside monitor and a central monitoring station. Note that as shown in FIG. 16, there can be multiple pathways for the lactate data to be received by various computing devices 120, including via several different external transmitters 122 and/or reader devices 124. These pathways can also update dynamically as the patient moves from different locations as needed to ensure lactate data is properly transferred as needed.

Remote computing devices 120 are configured to receive lactate data from continuous lactate monitor 110, including historical and real-time data. Remote computing devices 120 can be further configured to analyze the lactate data from continuous lactate monitor 110. Remote computing devices 120 can also be configured to display the lactate data from continuous lactate monitor 110. Remote computing devices 120 can also include alarms and analytical metrics of the data. The analytical metrics can include, for example, any combination of minimum lactate value, maximum lactate value, average lactate value, median lactate value, rate of change of any of the prior metrics, measures of the variability of the lactate values, time spent above a lactate limit over a certain time period, expressed in hours/minutes or percentage of time, and other suitable calculated metrics. The analytical metrics can be calculated over predetermined or customized time periods. Corresponding alarms can also be set with predetermined lactate data limits, such as a predetermined maximum, average, or minimum lactate values, or with customized limits. Certain alarms may also have tiered values. For example, a maximum lactate value of 2 mmol/L may trigger a first tier high lactate alarm (e.g., “lactate limit”), while a value over 4 mmol/L may trigger a second tier alarm that has a higher priority than the first tier (e.g., “high lactate limit”). Alarms may also be tailored to events. For example, an alarm may be triggered if lactate levels are not dropping after a procedure, or not dropping by a desired amount in a given time (i.e., a desired rate of decrease). Alarms may also require multiple conditions to trigger. For example, a high lactate alarm may also require the high lactate over a certain time period. For example, the high limit alarm may only be triggered if lactate is above 2 mmol/L for one hour. There may be multiple variants of these multi-limit alarms for a single value. Using the high limit alarm above as an example, there may additionally be an alarm that triggers if the value is above 4 mmol/L for fifteen minutes. Alarms may also vary based on patient condition. For example, if a patient is stable then the alarm conditions may be more relaxed to reduce false alarms. A deteriorating or critically ill patient may have strict alarm conditions to notify HCPs of any changes more quickly. Other examples of varying alarm conditions being tailored to the patient can be alarm conditions based on the patient's medical history, including any current medical treatments or diseases. For example, certain medical treatments or disease history may warrant stricter alarm conditions because of potential interactions between excess lactate and the treatment/disease.

Remote computing devices 120 can be further configured to compare the lactate data to an alarm condition. An alarm condition can be a single limiting value that is met, or can be a multi-step condition that can include, for example, a limiting value and a time component for which the limiting value is met. Remote computing devices 120 will transmit a notification or an alert if any alarm conditions are triggered. The notification or alert may inform the user that alarm conditions have been triggered. The notification or alert may be an audible and/or visual and/or haptic alert. The notification or alert can be transmitted by any suitable means, such as email, text message, internal message, application programming interface (API) or other data integration modality. Notifications and alerts can be stored in the patient's electronic health record. Notifications and alerts can be transmitted at the remote computing device and additionally be transmitted to one or more separate remote computing devices. Any combination of these techniques is possible. For example, local audible/visual notification on remote computing device 120 is possible, while simultaneously a notification can also be sent via email to a second remote computing device 120. This can be useful if an alarm condition is triggered in a home environment because the patient can be notified locally at their remote computing device 120 while the patient's HCP can also be notified by a separate message (e.g., an email) at their remote computing device. The alarms and/or notifications can be transmitted to any number of recipients, including various HCPs that are responsible for the patient, alerting systems (such as a central monitoring system at a hospital), patient caregivers, and the patient. This group can be dynamically updated as the patient's status changes. For example, when the patient is transferred to different departments in a hospital (e.g., ICU versus general floor), the list of recipients may change to accommodate different HCPs. This change can be made manually by altering settings in an application. This change can also be made automatically as part of the association and activation process of continuous lactate monitor 110. Each activation may have a unique default list of users to receive alarms that is prepopulated.

The use of continuous analyte data provides the capability to have real-time adjustments and configurability to alerts and notifications. As one example, threshold conditions associated with trends (e.g., an upward trend of 2.0 mmol/L or 4.0 mmol/L) may be used to trigger alerts and notifications. Certain recipient devices may be configured to enable (or prevent) the ability to toggle the threshold setting on and off and configure the threshold setting. Other trigger conditions may be associated with rate of change, threshold rate of lactate clearance, a threshold period of time for the rate of change, time spent above a lactate limit, and rates of change before and after treatments.

Alerts and notification timing may also be adjusted and configured based on the real-time data. For example, lactate clearance may be included as part of the alerting criteria. There may be a threshold buffer time for an abnormal reading before an alert is triggered.

Recipients of alerts and notifications may also be changed such as having an audible setting, location-based settings (e.g., within the hospital such as HCPs or nurses), EHR-based notifications, text to device, and bedside notifications.

Continuous lactate monitoring system 100 provides the lactate and other patient medical information to a prediction model for generating a predicted patient outcome. The predicted patient outcome may be based on the lactate information and/or the medical information. The prediction model may be implemented in any combination of continuous lactate monitor 110 and remote computing device 120 (e.g., reader device 124 and/or remote server 126). Examples of a predicted patient outcome include, but are not limited to, upward or downward trends in patient condition (i.e., patient deterioration), patient response to potential treatment, length of stay following a procedure, risk of readmission following patient discharge, and indicators of a potential disease or condition. For example, the prediction model may be trained to predict the length of a patient's stay following a procedure based on the patient's continuous lactate information. As noted above, examples of potential disease or condition includes but is not limited to sepsis, acute heart failure, polytrauma, lung disease, liver disease, cancer, and major surgery recovery.

Remote server 126 may be implemented a cloud-based server (or network of distributed servers). Remote server 126 may further be configured to implement components for generating alerts and notifications based on received continuous lactate data. The component may be implemented as a machine learning model (or models) for processing the continuous lactate data and performing an alert and notification process that includes steps for determining one or more recipient devices for receiving an alert and/or notification, determining the content to be included in the alert and/or notification, determining the visual format of the content based on the recipient devices and content, and transmitting the alert and/or notifications.

Inputs to the machine learning model may be chosen based on ensuring accuracy and relevancy of generated alerts to the particular recipient (e.g., EHR system, web application). One input may include continuous lactate data which consists of real-time measurements from continuous lactate monitor 110. Another input includes patient demographics such as age, gender, and social determinants of health (SDoH), which can influence the interpretation of analyte data. Another input can include medical history, surgical history, and historical health data, for example, where previous records of the patient's lactate levels, along with outcomes of past interventions, enable the model to identify trends and patterns that are specific to the patient. Another input may include environmental and contextual data such as time of day, current context (e.g., home vs. hospital), discharge information, previous and ongoing treatments, and hospital environment settings. These data can affect lactate levels and are thus included to refine the model's predictions.

Output from the machine learning model is a specifically structured alert or notification that is customized based on the continuous lactate data and the recipients receiving the data. Examples of visually structuring the alert or notification include content explaining the alert and notification, severity level, actionable recommendations, and customized visual content. For severity level, the model may categorize the urgency of the alert based on one or more of the inputs, using thresholds trained from historical data to classify alerts into levels such as critical, urgent, or informational. Based on the severity and the patient's specific context, the model generates actionable recommendations, such as adjusting medication dosages, scheduling medical reviews, contacting emergency personnel. For customized visual content, the model, for each alert, may select visual content appropriate for the intended recipient. This might include graphs showing trends in lactate levels for doctors, color-coded alerts for patients on their applications, or detailed tabular data for monitoring services. Note that these graphs can be customized as desired in terms of data points displayed, time ranges, data trend indications (and corresponding time ranges), and similar data. There may be default graph settings that are prepopulated in the various systems.

The user may set preferred or customized alarm conditions, alerts and/or notifications. For example, each individual user may customize preferred alarm condition settings. User preferences can be stored on the memory of the remote computing device 120. User preferences can also be stored on remote server 126 and retrieved by the computing device 120, optionally using a data network.

The type of recipient devices may impact and shape the type of alerts and notifications (e.g., those generated by the machine learning model). Recipient type not only influences the immediacy and urgency of the response but also determines the nature and detail of the visual indications and information that are incorporated into each alert and notification. Tailoring alerts and notifications ensures that each recipient receives the most relevant and actionable data in a format that best supports their role in patient care.

For a web application, remote alerts need to be comprehensive yet quickly interpretable to facilitate swift decision-making. Remote computing device 120 may therefore, generate alerts with detailed graphs and trend analyses. The graphs and trend analyses can highlight critical changes in lactate levels. The remote computing device may further suggest potential causes. These visual representations may be accompanied by a summary of the patient's current condition and optionally a comparison with previous similar cases, aiding staff in assessing the situation rapidly.

For an EHR system, the focus may be on documentation and long-term trend monitoring rather than immediate action. Alerts and notifications may be integrated as part of the ongoing record, with each notification including a timestamp, the measured lactate levels, and any actions taken or recommended. Visual content for the EHR may be more static, often presented in a format for providing long-term and short-term trends and providing access to an increased amount of visualization options which can be presented for display to understand the patient's history and adjust treatment plans accordingly.

For an HCP application, alerts and notifications may be designed to prompt immediate and informed medical intervention. Remote computing device 120 may provide alerts and notifications with high-resolution charts and/or predictive models that forecast the potential progression of the patient's condition based on the current lactate data. The application might also receive alerts with embedded links to the patient's EHR, for example, including past and current treatments, and optionally access to the patient's current location.

For a caregiver application, alerts and notifications may be simplified to ensure clarity and prevent unnecessary anxiety. These notifications often use color-coded systems—such as green, yellow, and red—to indicate the state of a patient's health. Accompanying text is written in layman's terms, with straightforward recommendations like “Rise in lactate due to activity, please rest” or “Time for your medication.” Other accompanying text, also written in layman's terms, may ask the caregiver to input additional information like “Please select other symptoms being experienced by the patient.” Visuals for caregiver applications may be customized with icons or simple graphs to show trends without overwhelming them with data.

For a monitoring service, which operates continuously, alerts and notifications may include a combination of detailed and summarized alerts. Third party monitoring services may receive comprehensive data, for example, including all past alerts, current analyte levels, and predictive insights from the model. Visual components in these alerts may be configured for quick scanning, and, for example, may include both detailed charts and summary bullet points. The monitoring service may be configured to perform real-time monitoring of the received continuous analyte data and generate customized visual elements for alerts and notifications based on the analyzed continuous analyte data and the intended recipient devices for the alerts and notifications.

Remote server 126 may store continuous analyte (e.g., lactate) data. Analyte data may be stored in a structured format. Analyte data may be stored within a secure database. The continuous analyte data may include metadata to allow for the traceability of the continuous analyte data and its accurate pairing with the associated patient. Examples of recipients include but are not limited to web applications, electronic health record systems, an application customized for use by a health care provider (e.g., the application may be secured from use by anyone other than the health care provider), an application associated with a dedicated caregiver (e.g., a user that is specified by the health care provider or the patient), and a third-party platform such as a remote monitoring system.

Remote server 126 may configure alerts/notifications based on the recipients that may have different access privileges to the continuous lactate data. For example, web applications may be configured with access privileges for the continuous lactate data, such as only having access to real-time and historical lactate values and calculated metrics (e.g., lactate rate of change). A web application may be configured to receive alerts both within the application and outside of the application (e.g., email, SMS). Electronic health record (EHR) systems may be configured to allow for dynamic navigation of stored data (such as using a flowsheet). Dynamic navigation may enable viewing of real-time lactate values and calculated metrics which may be provided at a frequency that is established by provider or health system workflows. Based on its access to additional user historical data EHR systems may be configured with additional alerts and notifications, such as popup alerts, abnormal result flags, and messaging-center messages. The additional alerts and notifications may be customized based on the user data and/or settings established by the HCP or other authorized user.

An HCP application may be configured with access privileges to not only view real-time lactate values and calculated metrics, but may have permissions for communicating with EHR systems and other applications (i.e., other HCP applications, other caregiver applications), and generating recommendations to be included in alerts and notifications based on those access privileges. A caregiver application may be configured with more limited access privileges compared to the HCP application. Alerts and notifications for the HCP and caregiver applications may be provided both within the respective applications as well as outside of the application, based on settings or permissions established for the particular continuous lactate data and the user of the application. A third-platform system, such as a remote monitoring system may integrate continuous lactate data into third-party visualizations based on access privileges granted to the third-party platform system.

Output of the prediction model may be used by continuous lactate monitoring system 100 to detect signs of deterioration in the patient. For example, a prediction model may be implemented in any combination of remote computing device 120 (e.g., reader device 124 and/or remote server 126). Each device may be configured to generate a predicted patient outcome based on received lactate information from continuous lactate monitor 110 associated with one or more patients. In some aspects, the device may also receive medical information associated the one or more patients and may be further configured to generate the predicted patient outcome based on both the received lactate information and the medical information. In some aspects, detection may be based on comparing the predicted patient outcome to predefined threshold values and signs of deterioration may be determined based on the predicted patient outcome exceeding one or more predefined threshold values. In some aspects, the predicted patient outcome may be implemented as any combination of a number value (e.g., between one and a hundred), a text message (e.g., “suspected sepsis” or “patient vitals deteriorating”), or a visual display such as a trend graph or a table (e.g., showing patient vitals including the continuously monitored lactate information).

Alerts and notifications can also include the predicted patient outcome. An alert differs from a notification with regard to any action that may be required based on the predicted patient outcome. An alert may require an immediate action. For example, an alert may require an immediate treatment to be administered, either manually by a caregiver or automatically by a medical device that is already connected to the patient. The alert may include one or more treatment recommendations for addressing the predicted patient outcome. In contrast, a notification may simply provide the predicted patient outcome and does not require any immediate action or treatment. Accordingly, alerts and notifications may be used based on a severity of the predicted patient outcome. Continuous lactate system 100 may generate the treatment recommendations based on the predicted patient outcome. An alert and/or notification may be generated to notify one or more recipients of the patient's predicted outcome.

Any remote computing devices 120 discussed above can include these data metrics and alarms. For example, the data metrics and alarms may be integrated into the patient's electronic health record such that they are available for access by an HCP. Data applications running on an existing computing device (e.g., a mobile phone or tablet) can also access the data metrics and alarms by effectively turning those existing computing devices into reader devices 124. There may be applications specific to the user. For example, in some embodiments there may be an HCP application and a caregiver application. Each application may require login by the user (e.g., the HCP or caregiver/patient) before accessing any data. These applications may have different data presentations depending on the context and target user. For example, the caregiver application may have a simplified and more user-friendly display of the data, while the HCP application may include more details (e.g., more data and data metrics) by default. Some users may have more restricted access to display of the data. For example, patient or caregiver displays may be restricted in what data is available to display. These restrictions may be managed or adjusted by, for example, the response HCP. These displays can be customized by each individual user, in terms of, for example, what data to display and/or the alarm condition settings to apply. User preferences can be stored on the memory of the remote computing device 120. User preferences can also be stored on remote server 126 and retrieved by the computing device 120, optionally using a data network. For example, the user preferences can be stored in the user's account and applied when the user logs in to remote computing device 120.

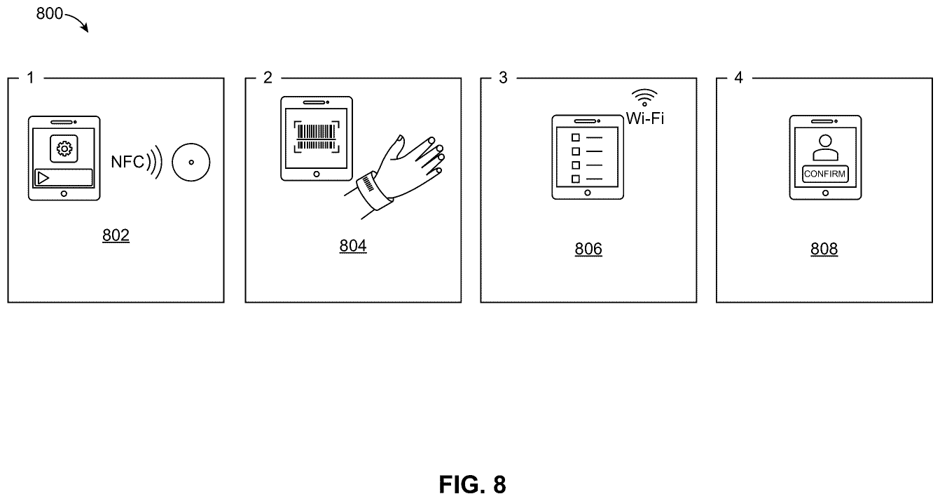

FIG. 8 shows an example of a hospital workflow 800 where the continuous lactate monitor 110 is integrated into Admission, Discharge, and Transfer (“ADT”) system. In an initialization step 802 continuous lactate monitor 110 is initialized by using wireless communication between an application on reader device 124 and continuous lactate monitor 110. As shown, the communication may be via a short-range wireless protocol such as NFC, but other communication protocols can be used, as discussed above. In an identification step 804 patient-specific information is captured to identify the patient. As shown, this can be accomplished by scanning a barcode or similar identifier physically attached or located with the patient using a suitable scanner in reader device 124. Alternatively, the patient information can be input manually into reader device 124. Further alternatively, the patient information may already be present in reader device 124 because of a login procedure or a filtered ADT feed producing a patient list on the reader device. In a step 806 the application uses the patient information to associate continuous lactate monitor 110 with the patient in the ADT system. Finally, in a step 808 the patient's identity is confirmed, for example by confirming patient information from the ADT system with the patient. This verification of patient information can occur as a positive patient identification process implementation. Continuous lactate monitor 110 will begin transmitting lactate data after it has been fully initialized and associated with the patient's information. The reader device 124 may only act to initialize continuous lactate monitor 110 and will not store any lactate data.

In some embodiments, hospital workflow 800 may be distributed across different devices within the distributed diagnostic system, such as one or more reader device 124 implemented in different physical locations of the hospital, such as the emergency room, ICU, and general floor. One or more reader devices 124 may be configured with different settings (e.g., on-screen display, alert notifications, communication frequency, recipient devices, patient identifiers, HCP identifiers, device identifiers) based on their physical location within the hospital. For example, one or more reader devices 124 implemented in the emergency room may be configured with lower thresholds for alert notifications, higher frequency of communicating continuous analyte data to other devices, and different recipient devices associated with emergency room personnel.

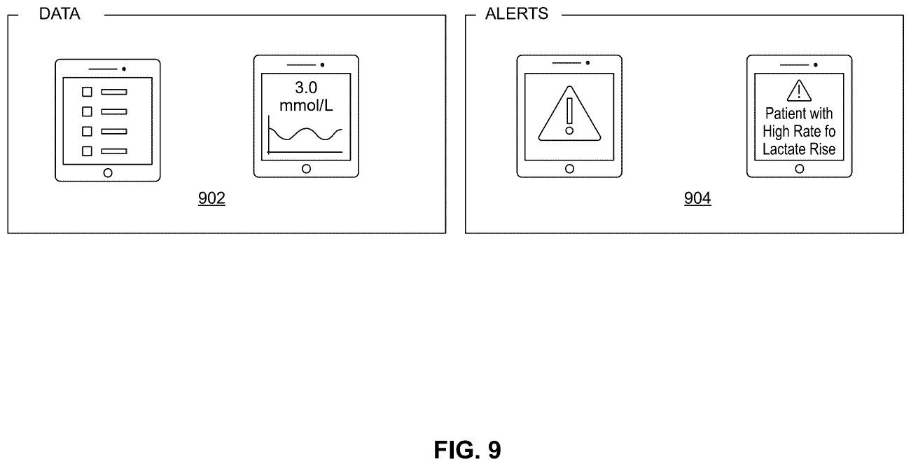

FIG. 9 shows some example data screens that can be accessed in any of the above applications on remote computing device 120. First, in a screen 902 a list of accessible patients can be presented. This list is populated by patients with available lactate data, and can be sorted in any desired manner, including with priority given to patients having active alarms triggered. In a screen 904 the patient's lactate data can be displayed. This can include numerical and/or graphical representations of current and/or historic lactate data. Note that the current lactate data can be requested on demand by the user of reader device 124. Screen 906 and 908 provide examples of alarm notification screens with visual indicators and textual descriptions of alarm notifications. All lactate data may be stored on remote server 126 and thus is not stored in the memory of the user's reader device 124.

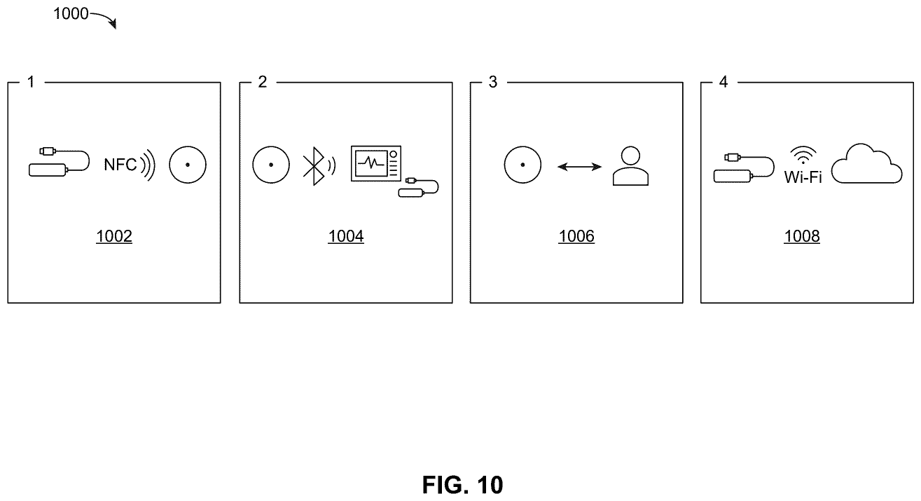

FIG. 10 shows a sensor initialization method 1000 in a hospital setting. In an initialization step 1002, continuous lactate monitor 110 is initialized using the adaptor embodiment external transmitter 122. This can be beneficial because no separate reader device 124 (or similar device) is needed because external transmitter 122 is typically already present in the patient's room being attached to an existing computing device (e.g., the patient's bedside monitor). This activation option may also be the preferred technique used in a patient telemetry environment (e.g., a hospital) because it can allow for easier integration of lactate data with the existing patient monitoring systems. Continuous lactate monitor 110 may be paired directly with external transmitter 122 instead of reader device 124. External transmitter 122 may also be configured to allow continuous lactate monitor 110 to late-join the relevant data network. In a step 1004 data from continuous lactate monitor 110 is captured by external transmitter 122 and is transmitted to the existing computing device. In a step 1006 continuous lactate monitor 110 is associated with the patient automatically because the patient information is already present on the existing computing device. For example, the existing computing device (e.g., the patient bedside monitor) already is associated with and has access to the patient's information, including a patient identifier. Connecting external transmitter 122 to the existing computing device allows the lactate data to be associated with the patient information (e.g., the patient identifier) already present. Finally, in a step 1008 lactate data is transmitted to other remote computing devices 120 as discussed above. Note that in telemetry environments, transmission of all lactate data from continuous lactate monitor 110 may pass through external transmitter 122 only. Any reader devices 124 or displays 128 will receive lactate data through suitable data networks (e.g., a hospital Wi-Fi network) after the lactate data has been passed to remote servers 126 for access.

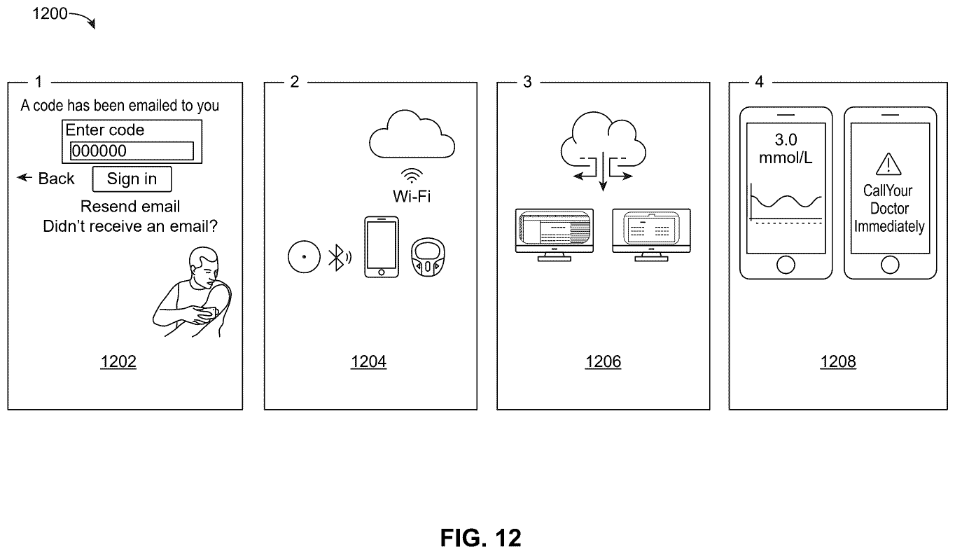

FIG. 12 shows an example of a home-based activation and monitoring process 1200. In an initialization step 1202 an account is created for the patient by the HCP. This can occur with or without the patient physically present. The patient can then access their account using their email address, a custom login code, or a custom link. Upon accessing the account created by the HCP, the patient can complete account setup and establish a password for the account. In some embodiments, the patient can self-create an account in lieu of the HCP creating an account, with the ability to notify an HCP of this patient-created account and/or to link this patient-created account to an HCP. The patient can then use the account to initialize continuous lactate monitor 110. In some embodiments, as discussed above, the patient uses reader device 124 to scan or electronically interface with continuous lactate monitor 110. Because the patient's account is already established and is used to initialize continuous lactate monitor 110, data from continuous lactate monitor 110 is already associated with the patient. In a step 1204 the lactate data from continuous lactate monitor 110 is transmitted to remote computing devices 120 via a suitable external transmitter 122 (e.g., the transmitter associated with the patient's reader device 124). This can include transmitting lactate data to remote server 126. In a step 1206, this data is then uploaded to the patient's electronic health record, or another data visualization platform, where it can be accessed by the HCP from a remote location. This enables the HCP and other services to remotely monitor the patient's information. In a step 1208 the patient can monitor their lactate data on reader device 124, which can deliver alerts to the patient as discussed above. Note that in the home setting, various other parties may receive the lactate data as part of a system to support the patient's health. These parties include third party monitoring services that can continuously monitor the lactate data and alert the patient if an anomaly is seen. This separate alerting can operate in addition to the automated alerts discussed above.

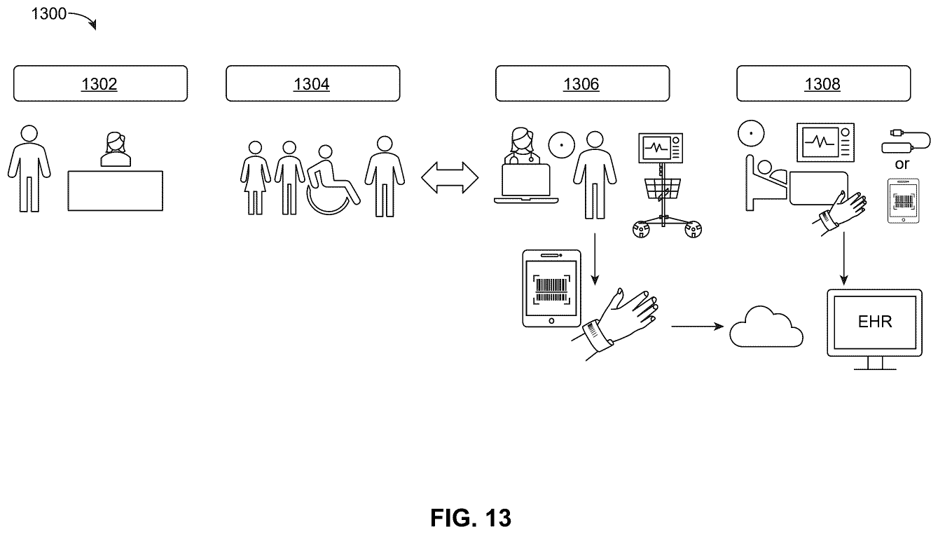

FIG. 13 shows an emergency department workflow 1300. In a step 1302 the patient presents at registration and received a wristband. In a step 1304 the patient waits in a waiting room to be seen. In a step 1306 the patient is seen by an HCP at a triage station. Here, the HCP can apply and activate continuous lactate monitor 110 using any of the techniques discussed above. The lactate data can be monitored as part of the triage procedure, with alerts and notifications being sent if abnormal data or conditions are detected. In a step 1308 the patient is admitted to a room for further treatment, with the monitoring of lactate data from continuous lactate monitor 110 being transferred seamlessly to the corresponding computing devices 120 as the patient transfers locations using the techniques discussed above.

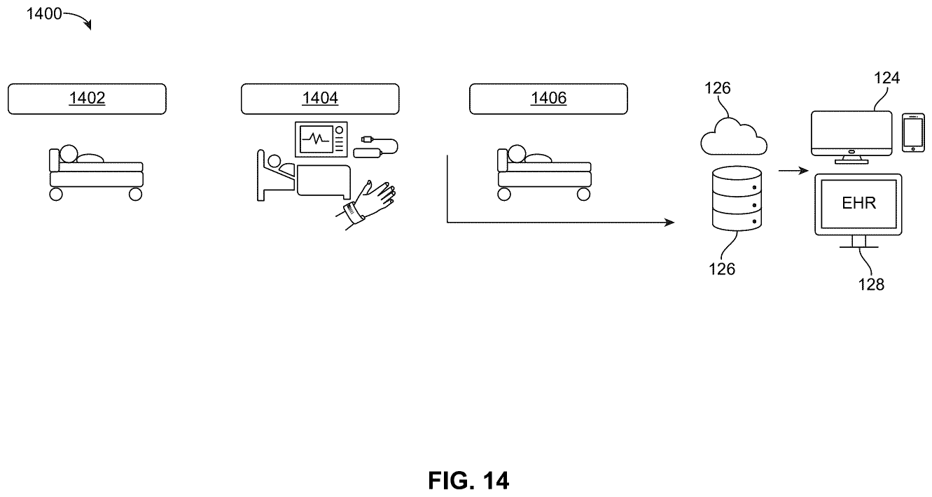

FIG. 14 shows an intensive care unit (“ICU”) workflow 1400. In a step 1402 the patient is transferred to the ICU from any suitable location (e.g., the emergency department or the general care floor). In a step 1404 the patient is admitted into a room. Continuous lactate monitor 110 is applied using the techniques above if the patient does not already have continuous lactate monitor 110. Alternatively, the existing continuous lactate monitor 110 is paired with computing devices 120 in the ICU room, if necessary. The lactate data can now be monitored on existing computing devices 120, with alerts and notifications being sent to the correct HCPs and other services because of the association process. In a step 1406 the patient is discharged with monitoring of lactate data continuing as needed using the techniques discussed above.

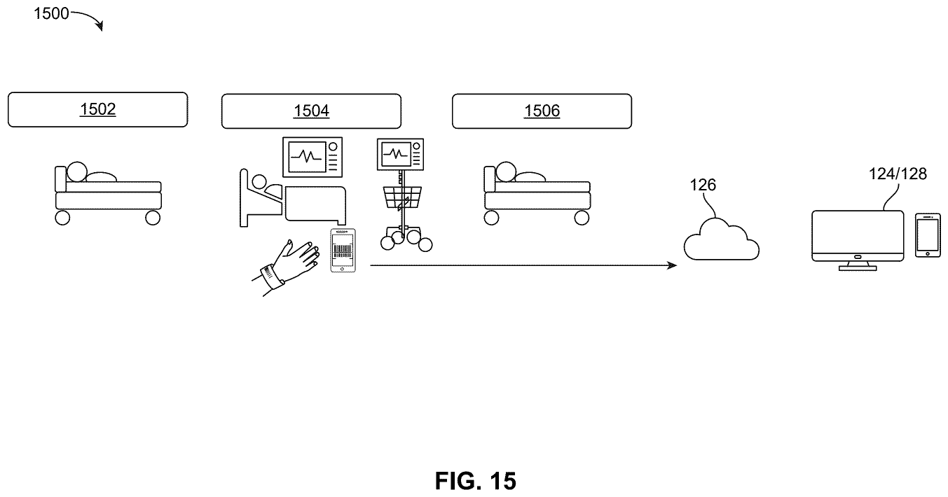

FIG. 15 shows a general care floor or ward workflow 1500. In a step 1502 the patient is transferred to the general care floor from any suitable location (e.g., the emergency department or the ICU). In a step 1504 the patient is admitted into a room. Continuous lactate monitor 110 is applied using the techniques above if the patient does not already have continuous lactate monitor 110. Alternatively, the existing continuous lactate monitor 110 is paired with computing devices 120 in the patient's room, if necessary. The lactate data can now be monitored on existing computing devices 120, with alerts and notifications being sent to the correct HCPs and other services because of the association process. In a step 1506 the patient is discharged with monitoring of lactate data continuing as needed using the techniques discussed above.

It is to be appreciated that the Summary and Abstract sections may set forth one or more but not all exemplary embodiments of the present invention as contemplated by the inventor(s), and thus, are not intended to limit the present invention and the appended claims in any way.

The foregoing description of the system and methods will so fully reveal the general nature of the invention that others can, by applying knowledge within the skill of the art, readily modify and/or adapt for various applications the systems and methods, without undue experimentation, without departing from the general concept of the present invention. Therefore, such adaptations and modifications are intended to be within the meaning and range of equivalents of the disclosed invention, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described examples, but should be defined only in accordance with the following claims and their equivalents.

The following is a numbered list of clauses of the invention.

Clause 1: A continuous lactate monitoring system, comprising: a continuous lactate monitor connected to the patient, the continuous lactate monitor configured to generate lactate data based on measured lactate levels of the patient, the continuous lactate monitor having a transmitter configured to transmit the lactate data; an external transmitter that is configured to receive the lactate data from the transmitter, a computing device connected to the continuous lactate monitor through the external transmitter, wherein the computing device includes a processor and memory, the memory storing instructions that when executed by the processor cause the processor to: receive lactate data from the continuous lactate monitor; analyze the lactate data to compare the data to an alarm condition; and transmit a notification and/or an alert if the lactate data triggers the alarm condition.

Clause 2. The system of clause 1, wherein the memory includes further instructions that cause the processor to transmit a notification and/or an alert to a second computing device when the alarm condition is triggered.

Clause 3. The system of clause 1 or 2, wherein the alert is an audible and/or visual alert.

Clause 4. The system of any preceding clause, wherein the notification is transmitted by email, text message, or internal message.

Clause 5. The system of any preceding clause, wherein the memory includes a user preference associated with a user of the computing device, optionally wherein the user preference is related to the alarm condition.

Clause 6. The system of any preceding clause, wherein the user preference is also stored on a remote server such that the user preference can be retrieved by the computing device, optionally wherein the user preference is retrieved using a data network.

Clause 7. The system of any preceding clause, wherein the memory includes further instructions that cause the processor to transmit the lactate data to a remote server, optionally using a data network.

Clause 8. The system of any preceding clause, further comprising a second computing device configured to receive the lactate data from the remote server.

Clause 9. A method of monitoring lactate in a patient, the method comprising: providing a continuous lactate monitor to be connected to the patient; generating lactate data based on measuring the patient's lactate levels using the continuous lactate monitor; transmitting the lactate data to a computing device using an external transmitter connected to the continuous lactate monitor; analyzing the lactate data to compare the data to an alarm condition; and transmitting a notification and/or an alert if the lactate data triggers the alarm condition.

Clause 10. The method of clause 9, wherein the steps of analyzing the lactate data and transmitting a notification and/or an alert are performed by the computing device.

Clause 11: The method of clause 9 or 10, further comprising transmitting a notification to a second computing device when the alarm condition is triggered.

Clause 12. The method of any of clauses 9 to 11, further comprising storing a user preference associated with a user of the computing device in a memory of the computing device, optionally wherein the user preference is related to the alarm condition.

Clause 13. The method of clause 12, further comprising storing the user preference on a remote server such that the user preference can be retrieved by the computing device, optionally wherein the user preference is retrieved using a data network.

Clause 14. The method of any of clauses 9 to 13, further comprising transmitting the lactate data from the computing device to a remote server, optionally using a data network.

Clause 15. The method of clause 14, further comprising receiving the lactate data from the remote server at a second computing device.

Clause 16. The system of any of clauses 1 to 8 or the method of any of clauses 9 to 15, wherein the computing device is a remote computing device.

What is claimed is:

1. A continuous lactate monitoring system, comprising:
a continuous lactate monitor, the continuous lactate monitor configured to generate lactate data based on measured lactate levels, the continuous lactate monitor having a transmitter configured to transmit the lactate data;
an external transmitter that is configured to receive the lactate data from the transmitter by wireless transmission; and
a computing device connected to the continuous lactate monitor through the external transmitter, wherein the computing device comprises memory that contains instructions that cause the computing device to:
receive lactate data from the continuous lactate monitor using the external transmitter;
analyze the lactate data to compare the lactate data to a predetermined lactate data condition stored in the memory; and
transmit a notification based on the analysis of the lactate data and the predetermined lactate data condition to a group of recipients, the group of recipients based at least in part on a patient's health status or a patient's physical location,
wherein at least one of the external transmitter and the computing device are configured to activate the continuous lactate monitor prior to transmission of the lactate data.

2. The system of claim 1, wherein the instructions further cause the computing device to transmit the notification and/or an alert to a second computing device when the predetermined lactate data condition is triggered.

3. The system of claim 1, wherein the alert is an audible and/or visual alert.

4. The system of claim 1, wherein the notification is transmitted by email, text message, or internal message.

5. The system of claim 1, wherein the memory includes a user preference associated with a user of the computing device, optionally wherein the user preference is related to the predetermined lactate data limit.

6. The system of claim 5, wherein the user preference is also stored on a remote server such that the user preference can be retrieved by the computing device, optionally wherein the user preference is retrieved using a data network.

7. The system of claim 1, wherein the memory includes further instructions that cause the processor to transmit the lactate data to a remote server, optionally using a data network.

8. The system of claim 7, further comprising a second computing device configured to receive the lactate data from the remote server.

9. The system of claim 1, wherein the external transmitter is configured to activate the continuous lactate monitor.

10. A method for continuously monitoring an analyte, comprising:
activating a continuous lactate monitor using an external transmitter;
connecting the external transmitter to a computing device;
receiving patient information from the computing device at the external transmitter;
automatically associating the continuous lactate monitor with the patient information;
receiving lactate data from the continuous lactate data at the computing device using the external transmitter; and
transmitting the lactate data from the computing device to a second computing device connected to the computing device.

11. The method of claim 10, further comprising:
analyzing the analyte data to compare the data to a predetermined lactate data condition; and
transmitting a notification and/or an alert when the analyte data triggers the predetermined lactate data condition.

12. The method of claim 11, further comprising transmitting a notification to the second computing device when the predetermined lactate data condition is triggered.

13. The method of claim 10, further comprising storing a user preference associated with a user of the computing device in a memory of the computing device, optionally wherein the user preference is related to the predetermined lactate data limit.

14. The method of claim 13, further comprising storing the user preference on a remote server such that the user preference can be retrieved by the computing device, optionally wherein the user preference is retrieved using a data network.

15. The method of claim 10, further comprising transmitting the analyte data from the computing device to a remote server, optionally using a data network.
